(19) United States
(12) Patent Application Publication
(10) Pub. No.: US 2022/0008926 A1
(43) Pub. Date: Jan. 13, 2022

(54) High-Throughput Microfluidic Isolation of Single Particles

(71) Applicant: The Penn State Research Foundation, University Park, PA (US)

(72) Inventors: Craig Eugene Cameron, State College, PA (US); Wu Liu, State College, PA (US)

(73) Assignee: The Penn State Research Foundation, University Park, PA (US)

(21) Appl. No.: 17/367,827

(22) Filed: Jul. 6, 2021

Related U.S. Application Data
(60) Provisional application No. 63/049,351, filed on Jul. 8, 2020.

(51) Int. Cl. B01L 3/00; G01N 1/40

(52) U.S. Cl. CPC B01L 3/502761; G01N 1/405; B01L 2300/16; B01L 2200/0652; B01L 2300/168

(57) ABSTRACT

The present invention provides microfluidic devices capable of sequestering single particles in individual microchambers and isolating the particles from one another. The devices provide a plurality of channels fluidly connected to a plurality of microchambers. A fluid suspension comprising particles of interest can be passed through the devices in a first direction to sequester single particles in each microchamber. An isolating fluid can be passed through the devices in a second, reverse direction to isolate the particles from one another. The devices can selectively isolate several particles in each microchamber.

### CROSS REFERENCE TO RELATED APPLICATIONS

This application claims the benefit of U.S. Provisional Application Ser. No. 63/049,351, filed on 8 Jul. 2020, which is incorporated herein by reference in its entirety as if fully set forth below.

### STATEMENT REGARDING FEDERALLY SPONSORED RESEARCH OR DEVELOPMENT

This invention was made with government support under Grant No. AI120560 awarded by the National Institutes of Health. The Government has certain rights in the invention.

### FIELD OF INVENTION

This invention generally relates to microfluidic devices. More particularly, this invention relates to microfluidic devices capable of sequestering single particles in individual microchambers and isolating particles from one another.

### BACKGROUND

The capability of a virus to escape natural immunity and evolve drug resistance stems from the molecular heterogeneities in viral and cellular genomes (Combe M et al., Cell host & microbe, 2015, 18(4):424-432; Heldt F S et al., Nature communications, 2015, 6:8938; Bolton D L et al., PLoS pathogens, 2017, 13(6):e1006445). By promoting understanding in how genetic diversities contribute to manifested virus-host interactions, recently arisen single-cell virology aims to fill in this “blind spot” of traditional assays and aid in effective treatment of viral infectious diseases (Russell A B et al., Elife, 2018, 7:e32303; Xin X et al., Journal of virology, 2018, 92(9):e00179-18; Chen Z et al., Frontiers in microbiology, 2017, 8:1831; Cristinelli S et al., Current opinion in virology, 2018, 29:39-50; Rato S et al., Virus research, 2017, 239:55-68). As single-cell analysis starts to open new vistas for virologists to explore, one of the bottlenecks remains in the lack of tools that enable large-scale, long-term, and real-time monitoring of viral replication and transmission. Specifically, the single-cells first need to be effectively isolated, as viruses could produce thousands of copies of themselves in a cell and spread to neighboring cells even before the infected cell lyses (Sattentau Q, Nature Reviews Microbiology, 2008, 6(11):815; Bird S W et al., Virology, 2015, 479:444-449).

Thus, there is a need in the art for a device that reliably captures and isolates particles, including cells, for individual analysis. The present invention satisfies this need.

### SUMMARY OF THE INVENTION

It is an object of the present invention to provide systems, devices, and methods to meet the above-stated needs. Generally, it is an object of the present invention to provide a delivery member for delivering and deploying an implantable medical device having a flexible distal portion.

In one aspect, the present invention relates to a microfluidic device for isolation of single particles, comprising: a planar substrate having an anterior end, a posterior end, a top surface, a bottom surface, and a thickness in-between the top and bottom surface; at least one anterior port and at least one posterior port extending from the top surface into the substrate; at least one channel embedded within the substrate; and at least one microchamber embedded within the substrate; wherein each anterior port is fluidly connected to a posterior port by the at least one channel; and wherein the at least one microchamber is connected to a channel by an anterior opening and at least one posterior opening.

In one embodiment, the at least one microchamber comprises a shape that tapers posteriorly towards the at least one posterior opening. In one embodiment, the at least one channel has a segment for each microchamber that is in direct alignment with the anterior opening of each microchamber.

In one embodiment, the at least one channel has a segment with a channel opening connected to each posterior opening of each microchamber by a channel branch. In one embodiment, the segment with the channel opening is aligned along a first axis and the channel branch is aligned along a second axis, such that the first axis and the second axis are substantially orthogonal to each other. In one embodiment, the at least one channel has a width between about 5 μm and 500 μm and a height or depth between about 5 μm and 50 μm. In one embodiment, the at least one microchamber has a length, a width, and a height or depth that is each between about 20 μm and 500 μm. In one embodiment, the anterior opening has a width between about 10 μm and 100 μm. In one embodiment, the at least one posterior opening has a width between about 1 nm and 100 μm.

In one embodiment, the substrate is at least partially transparent or translucent. In one embodiment, the at least one channel, the at least one microchamber, or both have one or more gradations within the thickness of the substrate. In one embodiment, the at least one channel, the at least one microchamber, or both have an inner surface further comprising a surface treatment.

In one embodiment, the surface treatment includes an extracellular matrix material selected from the group consisting of: collagen, fibrin, fibrinogen, thrombin, elastin, laminin, fibronectin, vitronectin, hyaluronic acid, chondroitin 4-sulfate, chondroitin 6-sulfate, dermatan sulfate, heparin sulfate, vixapatin (VP12), heparin, and keratan sulfate, proteoglycans, chitin, chitosan, alginic acids, alginates, and combinations thereof.

In one embodiment, the surface treatment includes a drug selected from the group consisting of: analgesics, anesthetics, antifungals, antibiotics, anti-inflammatories, nonsteroidal anti-inflammatory drugs (NSAIDs), anthelmintics, antidotes, antiemetics, antihistamines, anti-cancer drugs, antihypertensives, antimalarials, antimicrobials, antipsychotics, antipyretics, anti septics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, fluorescent nanoparticles such as nanodiamonds, sedatives, sulfonamides, stimulants, sympathomimetics, tranquilizers, urinary anti-infectives, vasoconstrictors, vasodilators, vitamins, xanthine derivatives, and combinations thereof. In one embodiment, the surface treatment includes a capture agent selected from the group consisting of: antibodies, antigens, aptamers, affibodies, proteins, peptides, nucleic acids, carbon nanotubes, nanowires, magnetic beads, and fragments thereof.

In another aspect, the present invention relates to a method of isolating single particles, comprising the steps of: providing a microfluidic device comprising a substrate having at least one anterior port fluidly connected to at least one posterior port by one or more embedded channels, the substrate further comprising one or more embedded microchambers fluidly connected to each channel by an anterior opening and at least one posterior opening; flowing a suspension fluid comprising at least one particle of interest into the at least one anterior port, such that a particle of interest enters at least one microchamber through the anterior opening and blocks the at least one posterior opening; and flowing an isolating fluid into the at least one posterior port, such that the isolating fluid occupies each channel and isolates each microchamber.

In one embodiment, the suspension fluid, the isolating fluid, or both are flowed using a positive pressure or a negative pressure. In one embodiment, the at least one particle of interest is selected from the group consisting of: cells, viruses, bacteria, amoeba, microparticles, nanoparticles, beads, microorganisms, vesicles, and fragments thereof. In one embodiment, the suspension fluid is selected from the group consisting of: water, cell growth media, serum, plasma, and oil. In one embodiment, the isolation fluid is selected from the group consisting of: oils, gels, liquid metals, liquid polymers, and glues.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 depicts an exemplary microfluidic device having 6000 microchambers along 60 channels.

FIG. 2 depicts a magnified view of an exemplary end port connected to several channels and microchambers.

FIG. 3 depicts a magnified view of an exemplary single microchamber in fluid connection with a channel.

FIG. 4 depicts a perspective three-dimensional view of an exemplary single microchamber in fluid connection with a channel.

FIG. 5 depicts exemplary photomasks designed for fabrication of the microfluidic devices. The top image is the channel layer and the bottom is the microchamber layer.

FIG. 6 is a flowchart depicting an exemplary method of isolating particles.

FIG. 7A through FIG. 7C depict the results of experiments demonstrating high-throughput generation of isolated single-cells for virological studies. (FIG. 7A) COMSOL simulation of the flow velocity field during a single-cell trapping step (top) and the afterwards isolation of the chambers by FC-70 oil (bottom). The oil fills the channel area without rushing into the microchambers. The depicted channels are 20 μm in depth and the chambers are 100 μm in depth. Infuse rate: 200 nL/min. (FIG. 7B) Single-cell occupancy higher than 80% can be achieved using HeLa S3 cell (average diameter: ˜16 μm) with a wide range of cell density and infuse rates. (FIG. 7C) Single HeLa S3 cells trapped in the microchambers.

FIG. 8 depicts the isolation of the microchambers on-chip by FC70 oil. The top channels were filled with Coomassie blue solution while FC70 oil was infused into the bottom channels. The color difference between the channels and the chambers demonstrates that the aqueous-solution-containing microchambers were isolated by the oil

FIG. 9 depicts the trapping of single HAP1 cells. Single HAP1 cells could be effectively trapped despite their smaller average diameter (˜10 μm) than HeLa cells.

FIG. 10 is a table listing the means and relative standard deviations (RSDs) of the parameters for cells infected by different viruses. GFP (mCherry): signal from cells solely infected by GFP (mCherry); Co-GFP (Co-mCherry): GFP (mCherry) signal from co-infected cells.

FIG. 11 depicts the distributions of (top left) maximum, (top right) slope, (bottom left) infection time, and (bottom right) start point for cells infected by coxsackievirus B3 (CVB3). Numerical values for experimental parameters are provided in FIG. 10.

FIG. 12 is a table listing the P-values between groups based on t-test for the CVB3 infection experiment listed in FIG. 10.

FIG. 13 depicts the distributions of (top left) maximum, (top right) slope, (bottom left) infection time, and (bottom right) start point for cells infected by poliovirus (PV). Numerical values for experimental parameters are provided in FIG. 10.

FIG. 14 is a table listing the P-values between groups based on t-test for the PV infection experiment listed in FIG. 10.

FIG. 15 depicts the correlations between the GFP signal and the mCherry signal for maximum, slope, infection time, and start point derived from single HeLa S3 cells co-infected by GFP- and mCherry-tagged CVB3 in (a) to (d), PV-WT in (e)-(h) or PV-G64S in (i)-(l). GFP and mCherry fluorescence intensities of each single cell were collected every 30 min from 1 hpi to 24 hpi, respectively.

FIG. 16 depicts the results of experiments demonstrating that PV exhibited wider variances in replication kinetic parameters than CVB3. Distributions of (top left) maximum, (top right) slope, (bottom left) infection time, and (bottom right) start point of mCherry-tagged virus infection are presented. The distributions of parameters for CVB3 are linearly normalized to have same means as those for PV. The means from raw data and RSDs are listed in FIG. 10.

FIG. 17A through FIG. 17D depict the results of cell pairing to investigate the spread of viral infection at the single-cell level. (FIG. 17A) Bright-field and fluorescent images showing the cell-pairs captured in the microfluidic chambers. Equal number of HeLa S3 cells were labelled respectively with Vybrant DiD (red) and DiO (green), mixed, and then infused to the microfluidic channels. (FIG. 17B) Optimization of the infuse rate and cell density for cell pairing. (FIG. 17C) Images showing different spread mode of PV infection when a PV-GFP-infected cell (pre-labelled with Vybrant DiD) and an un-infected cell were isolated in a microchamber and monitored for 24 hours. The donor cell would turn from red to yellow along with the replication of PV-GFP, and turn back to red when the cell lyses. (FIG. 17D) Nonlytic and lytic spread of PV infection could be distinguished based on the lysis of the donor cell and the start of viral replication in the recipient cell. For those donor cells didn't lyse by 24 hpi, 24.5 hpi is set as the lysis time here.

### DETAILED DESCRIPTION

The present invention provides microfluidic devices capable of sequestering single particles in individual microchambers and isolating the particles from one another. The devices provide a plurality of channels fluidly connected to a plurality of microchambers. A fluid suspension comprising particles of interest can be passed through the devices in a first direction to sequester single particles in each microchamber. An isolating fluid can be passed through the devices in a second, reverse direction to isolate the particles from one another. The devices can selectively isolate several particles in each microchamber.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%,±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Microfluidic Device for Particle Isolation

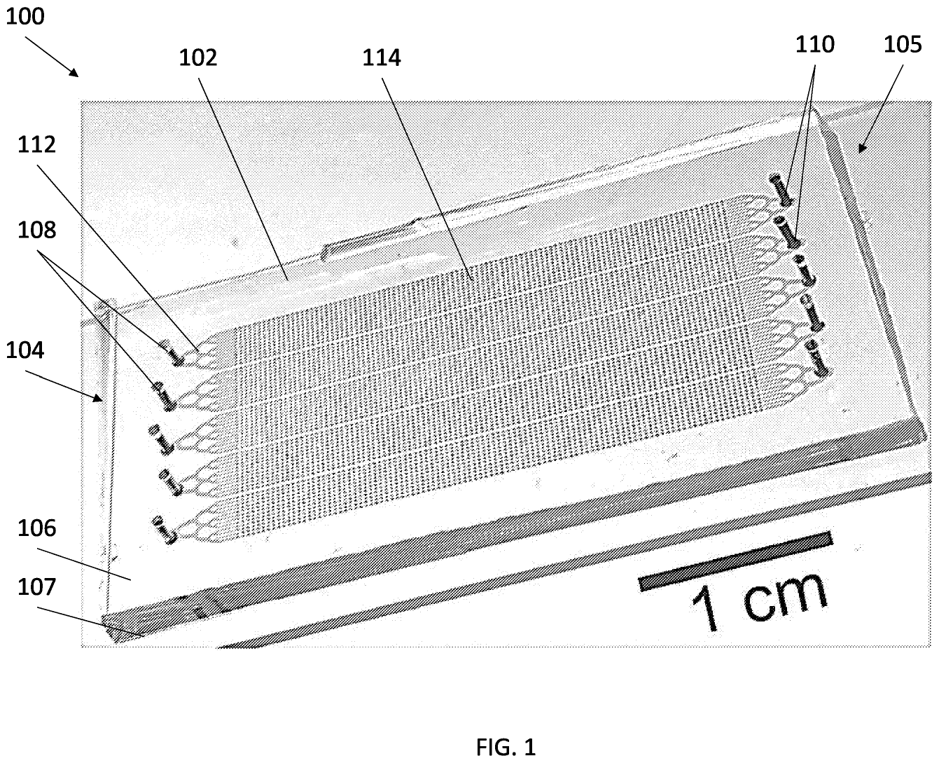

Referring now to FIG. 1, an exemplary microfluidic device 100 is depicted. Device 100 comprises a substrate 102 having an anterior end 104 and a posterior end 105. Substrate 102 comprises a top surface 106, a bottom surface 107, a thickness in-between, and at least one anterior port 108 and at least one posterior port 110 extending from top surface 106 into substrate 102, wherein each anterior port 108 is fluidly connected to a posterior port 110 by at least one channel 112 embedded within substrate 102. Each channel 112 is fluidly connected to at least one microchamber 114 embedded within substrate 102.

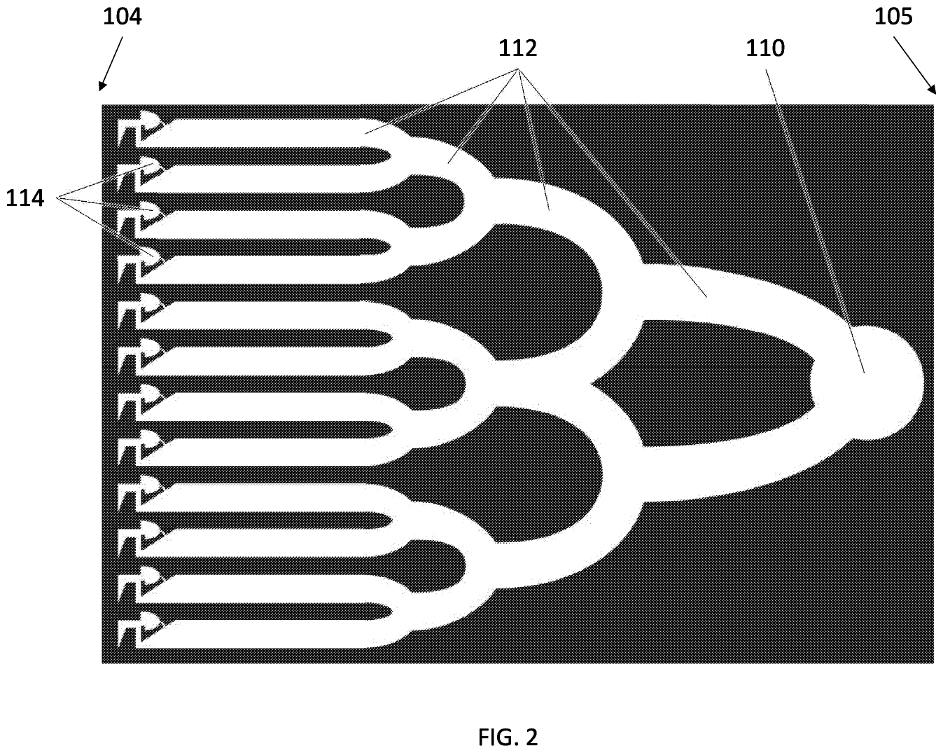

Referring now to FIG. 2, a magnified view of a port (here, posterior port 110) and a series of fluidly connected channels 112 is shown. A single port (anterior port 108 or posterior port 110) can be fluidly connected to one or more channels 112 to transfer and receive a fluid. While the embodiment depicted in FIG. 2 has a single posterior port 110 fluidly connected to twelve channels 112, it should be understood that any number of channels 112 can be connected to a port, including but not limited to 1, 2, 3, 4, 5, 6, 7, 8, 9, 10, 15, 20, 25, 30, 35, 40, 45, 50, or more channels 112. Visible on the left side of FIG. 2, each channel 112 is fluidly connected to at least one or more microchambers 114. The embodiment depicted in FIG. 1 has 100 microchambers 114 fluidly connected to each channel 112, but it should be understood that any number of microchambers 114 can be connected to a channel 112, including but not limited to 1, 10, 100, 1000, 10000, or more microchambers 114.

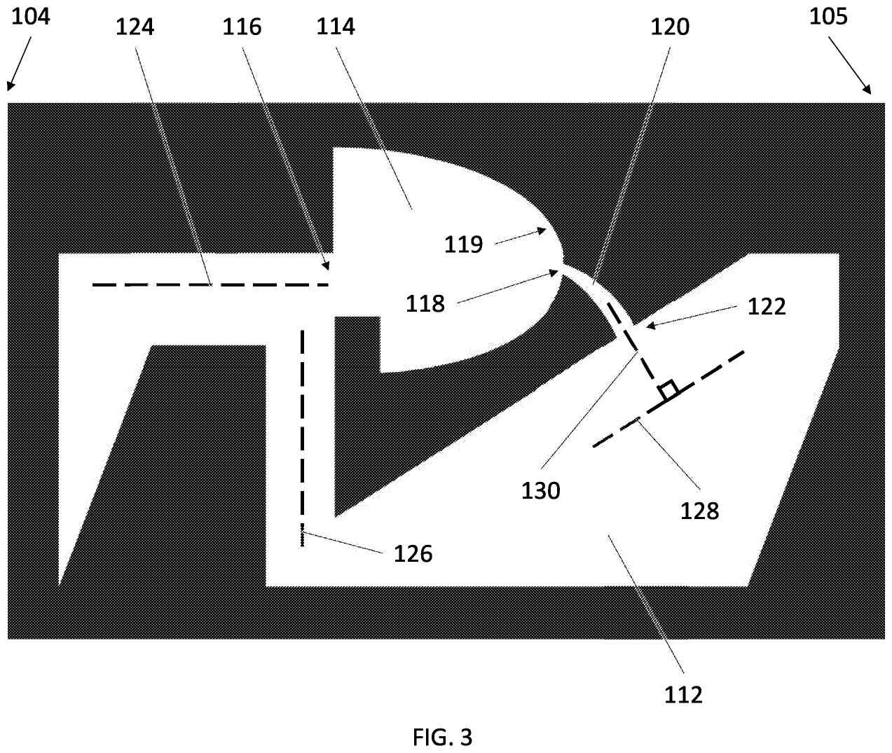

Referring now to FIG. 3, a magnified view of a microchamber 114 connected to a segment of a channel 112 is shown. Microchamber 114 is fluidly connected to channel 112 by at least two entryways, an anterior opening 116 and a posterior opening 118. While microchamber 114 is depicted in FIG. 3 as having a substantially bullet-like shape, it should be understood that microchamber 114 can have any suitable shape. In certain embodiments, microchamber 114 has at least one taper 119, wherein one or more posterior openings can be positioned at a narrow end of the at least one taper 119.

In some embodiments, channel 112 comprises a substantially linear path of repeating sections with segments of channel 112 aligned along a first axis 124, a second axis 126, and a third axis 128. First axis 124 can be aligned in an anterior-to-posterior direction in line with anterior opening 116. Second axis 126 can be aligned at an angle away from first axis 124, such as an angle between about 60° and 120° from first axis 124. Third axis 128 can be aligned at an angle away from second axis 126, such as an angle between about 30° and 90° from second axis 126. Channel 112 is also connected to posterior opening 118 by at least one channel branch 120 extending between posterior opening 118 and channel opening 122. In some embodiments, channel opening 122 is aligned along a fourth axis 130 that is substantially orthogonal to third axis 128.

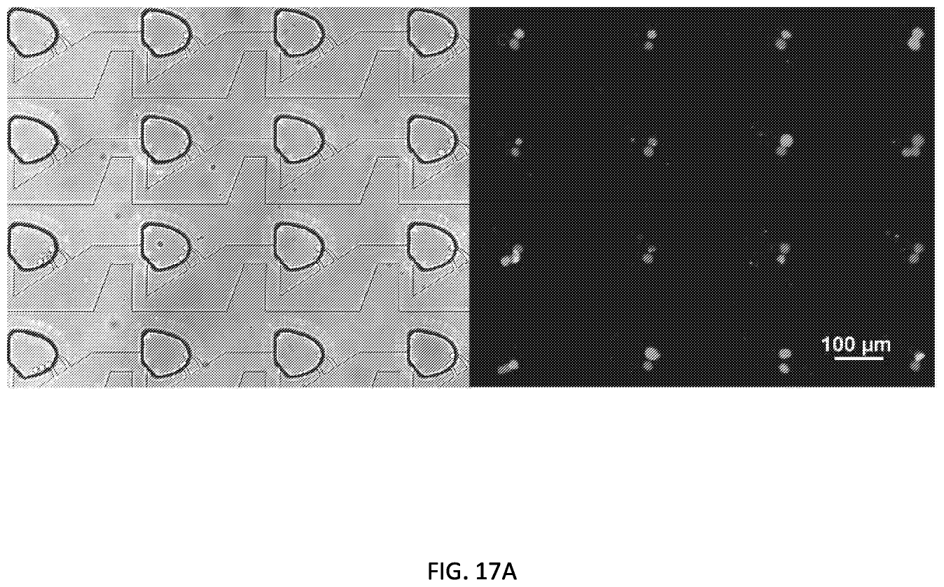

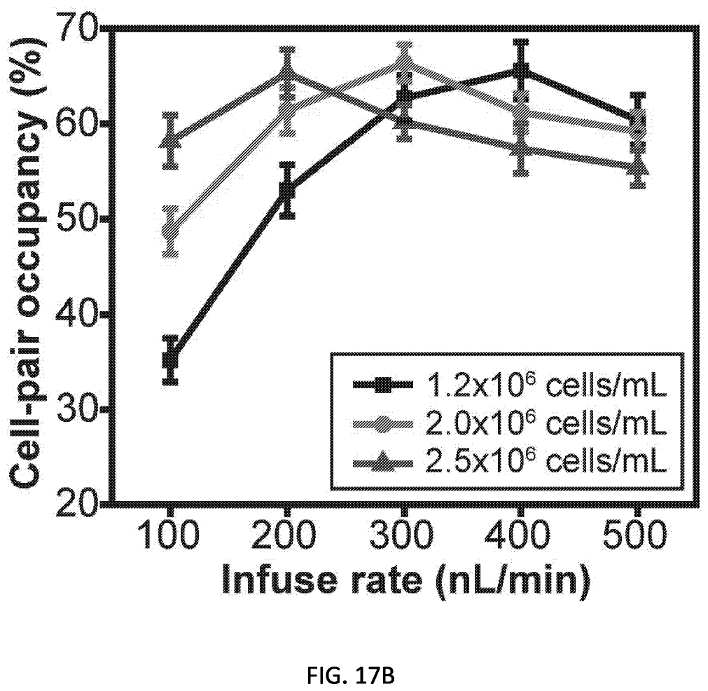

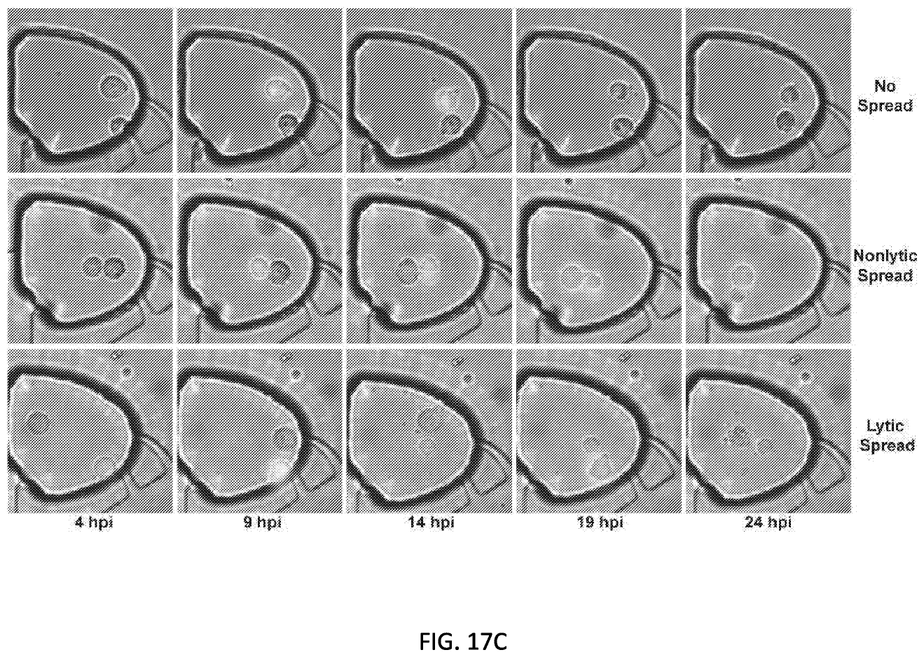

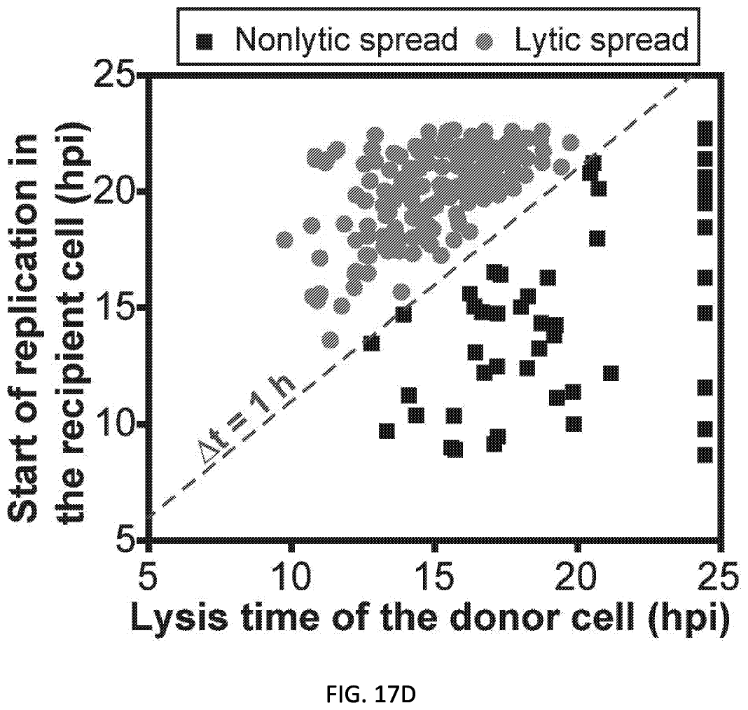

In various embodiments, each microchamber 114 can include a plurality of posterior openings 118, each connected to at least one channel opening 122 by a channel branch 120. For example, FIG. 17A and FIG. 17C each depict the isolation of two cells in a microchamber by way of two posterior openings connected to channel openings by channel branches. However, it should be understood that a microchamber 114 can support any number of posterior openings 118, including 1, 2, 3, 4, 5, 6, 7, 8, 9, 10, or more.

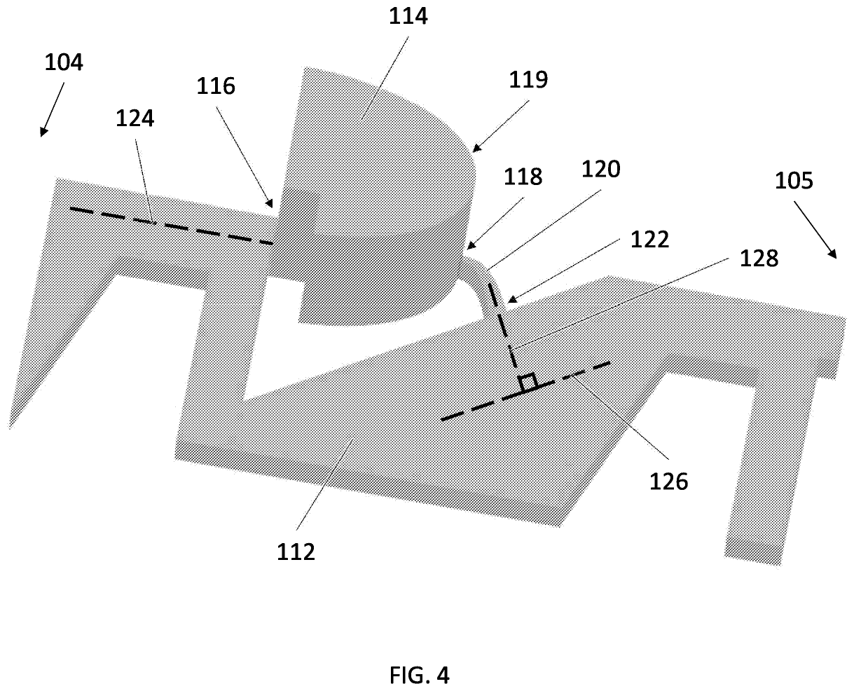

Device 100 can have any suitable dimensions. For example, substrate 100 can have any suitable length, width, and height (i.e., thickness) to fit the ports, channels, and microchambers, including but not limited to a length between about 1 cm to about 50 cm, a width between about 1 cm to about 50 cm, and a height or thickness between about 1 mm to about 10 mm. Each anterior port 108 and posterior port 110 can have a diameter between about 0.5 mm to about 15 mm. Channels 112 can have a width between about 5 μm and 500 μm and a height or depth between about 5 μm and 50 μm. Visible in FIG. 4, microchambers 114 can have a length, a width, and a height (i.e., depth) between about 20 μm and 500 μm each. Anterior opening 116 can have a width between about 10 μm 10 and 100 μm. Posterior opening 118 can have a width between about 1 nm and 100 μm. In various embodiments, the width of posterior opening 118 can be selected to be smaller than the width of a desired capture target. For example, a device 100 can be fabricated to have posterior openings 118 of width 4 μm to isolate desired particles having a width of 5 μm or more. In certain embodiments, microchannels 114 having more than one posterior opening 118 can each have posterior openings 118 of the same size or of different sizes to capture the same or different particles. Channel opening 122 can have a width between about 1 μm and 100 μm.

Device 100 is not limited in the three-dimensional arrangement of the ports, channels, and microchambers. While FIG. 4 depicts channels 112 and outlet 120 aligned along a single plane and being connected to microchamber 114 at the same elevation, channels 112, outlet 120, and microchamber 114 can each include one or more gradations through the thickness of substrate 102. For example, anterior opening 116 and posterior opening 118 can be positioned at any location along a height or depth of a microchamber 114, and channel 112 and outlet 120 can have a suitable angled or graded construction to connect to the anterior opening 116 and posterior opening 118. In some embodiments, anterior opening 116 is positioned at a higher plane than posterior opening 118. In another example, microchamber 114 can have a tapered height or depth similar to taper 119.

The microfluidic devices of the present invention can be constructed from any suitable material, such as metals and polymers including but not limited to: stainless steel, titanium, aluminum, poly(urethanes), poly(siloxanes) or silicones, poly(ethylene), poly(vinyl pyrrolidone), poly(-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO), polyorthoesters, polyether ether ketone (PEEK), polydimethylsiloxane (PDMS), and the like. In some embodiments, certain components or portions of certain components can be constructed from a transparent or translucent material. The devices can be made using any suitable method known in the art. The method of making may vary depending on the materials used. For example, devices substantially comprising a metal may be milled from a larger block of metal or may be cast from molten metal. Likewise, components substantially comprising a plastic or polymer may be milled from a larger block, cast, or injection molded. In some embodiments, the devices may be made using 3D printing or other additive manufacturing techniques commonly used in the art. In some embodiments, the methods can embed additional components, such as circuitry, electrodes, magnets, diodes, and the like, such that the resulting device can be electrified to support electroporation, photoporation, magnetic fields, and the like. In various embodiments, coatings, patterns, and other finely detailed features can be applied using techniques such as etching, lithography, deposition, spin coating, dip coating, and the like.

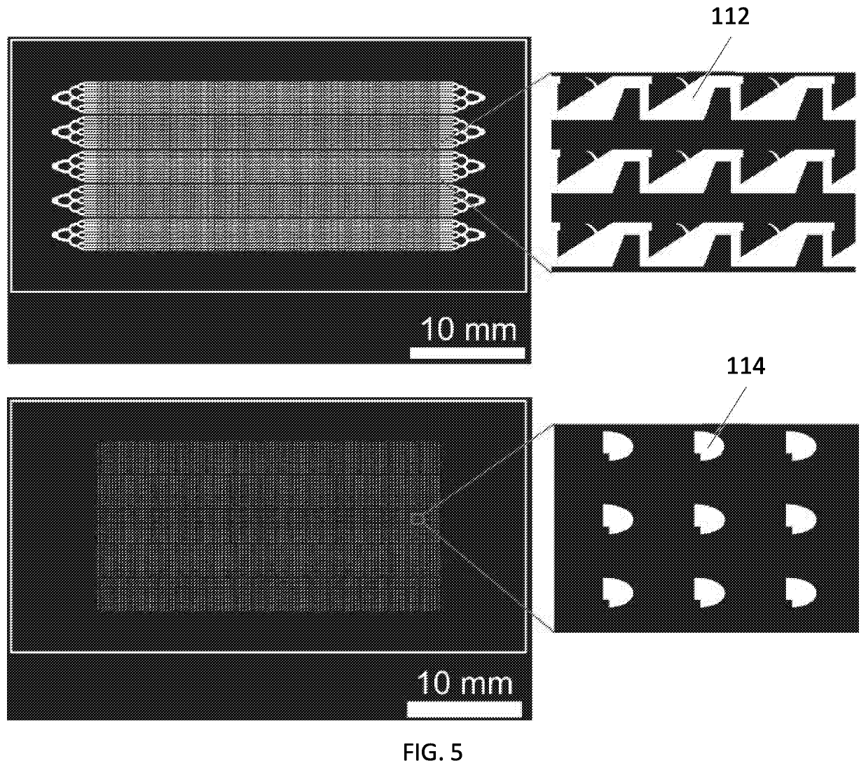

In certain embodiments, the devices are cast from molds. For example, FIG. 5 depicts designs for channels (top) and microchambers (bottom) for fabricating a positive mold through patterning with SU8 photoresist and multilayer lithography. For an exemplary device, the positive mold can comprise a channel layer having an exemplary 20 μm height and a microchamber layer having an exemplary 100 μm height. Curing a polymer within the mold would result in a substrate having a top surface with corresponding embedded channels having an exemplary 20 μm depth and microchambers having an exemplary 100 μm depth. Ports can be included in the mold or added afterwards by punching or milling. The top surface of the substrate can be sealed using a layer of metal, glass, or polymer. In various embodiments, the device can be cleaned or sterilized using any sterilization techniques, including but not limited to autoclaving, gamma ray sterilization, electron beam sterilization, and the application of any sterilizing gas, plasma, or solution, such as ethylene oxide, chlorine dioxide, hydrogen peroxide, oxygen plasma, and the like.

In certain embodiments, the devices can be subjected to one or more surface treatments. The application of the one or more surface treatments can facilitate the adherence and growth of cell lines. For example, the one or more surface treatments can include one or more extracellular matrix material and/or blends of naturally occurring extracellular matrix material, including but not limited to collagen, fibrin, fibrinogen, thrombin, elastin, laminin, fibronectin, vitronectin, hyaluronic acid, chondroitin 4-sulfate, chondroitin 6-sulfate, dermatan sulfate, heparin sulfate, vixapatin (VP12), heparin, and keratan sulfate, proteoglycans, and combinations thereof. Some collagens that may be beneficial include but are not limited to collagen types I, II, III, IV, V, VI, VII, VIII, IX, X, XI, XII, XIII, XIV, XV, XVI, XVII, XVIII, and XIX. These proteins may be in any form, including but not limited to native and denatured forms. In various embodiments, the one or more surface treatments can include one or more carbohydrates such as chitin, chitosan, alginic acids, and alginates such as calcium alginate and sodium alginate. These materials may be isolated from plant products, humans or other organisms or cells or synthetically manufactured.

In various embodiments, the surface treatments can include natural peptides, such as glycyl-arginyl-glycyl-aspartyl-serine (GRGDS), arginylglycylaspartic acid (RGD), and amelogenin. In some embodiments, the surface treatments can include sucrose, fructose, cellulose, or mannitol. In some embodiments, the surface treatments can include nutrients, such as bovine serum albumin. In some embodiments, the surface treatments can include vitamins, such as vitamin B2, vitamin Ad, Vitamin D, Vitamin E, and Vitamin K. In some embodiments, the surface treatments can include nucleic acids, such as mRNA and DNA. In some embodiments, the surface treatments can include natural or synthetic steroids and hormones, such as dexamethasone, hydrocortisone, estrogens, and its derivatives. In some embodiments, the surface treatments can include growth factors, such as fibroblast growth factor (FGF), transforming growth factor beta (TGF-β), and epidermal growth factor (EGF). In some embodiments, the surface treatments can include a delivery vehicle, such as nanoparticles, microparticles, liposomes, viral and non-viral transfection systems.

In various embodiments, the surface treatments can include one or more natural or synthetic drugs, including but not limited to: analgesics, anesthetics, antifungals, antibiotics, anti-inflammatories, nonsteroidal anti-inflammatory drugs (NSAID s), anthelmintics, antidotes, antiemetics, antihistamines, anti-cancer drugs, antihypertensives, antimalarials, antimicrobials, antipsychotics, antipyretics, anti septics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, fluorescent nanoparticles such as nanodiamonds, sedatives, sulfonamides, stimulants, sympathomimetics, tranquilizers, urinary anti-infectives, vasoconstrictors, vasodilators, vitamins, xanthine derivatives, and the like. The therapeutic agent may also be other small organic molecules, naturally isolated entities or their analogs, organometallic agents, chelated metals or metal salts, peptide-based drugs, or peptidic or non-peptidic receptor targeting or binding agents.

In various embodiments, the surface treatments can include one or more capture agents immobilized on an inner surface of the device. Capture agents have an affinity to analytes of interest and can be used for a number of purposes, including but not limited to detecting analyte presence, quantifying analyte amount, and filtering analytes from a solution. In some embodiments, certain capture agents employ nonspecific binding. In some embodiments, certain capture agents employ size-specific filtration and do not rely on binding. Contemplated capture agents include at least antibodies, antigens, aptamers, affibodies, proteins, peptides, nucleic acids, carbon nanotubes, nanowires, magnetic beads, and fragments thereof. The capture molecules can be provided in a uniform coating, in an array pattern, or in any shape or form desired.

### Methods of Use

The present invention also includes methods of using microfluidic devices for the isolation of single particles. As described elsewhere herein, the microfluidic devices of the present invention are capable of sequestering single particles from a fluid suspension in individual microchambers and isolating the particles from one another using an isolating fluid.

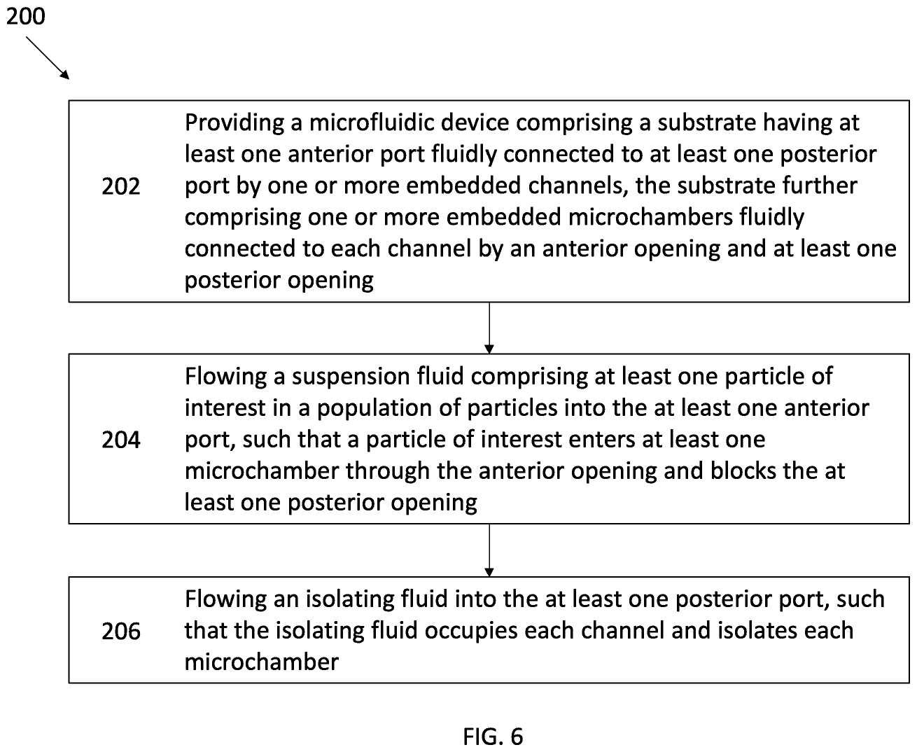

Referring now to FIG. 6, an exemplary method 200 of isolating single particles is depicted. Method 200 begins with step 202, wherein a microfluidic device is provided, the microfluidic device comprising a substrate having at least one anterior port fluidly connected to at least one posterior port by one or more embedded channels, the substrate further comprising one or more embedded microchambers fluidly connected to each channel by an anterior opening and at least one posterior opening. In step 204, a suspension fluid comprising at least one particle of interest in a population of particles is flowed into the at least one anterior port, such that a particle of interest enters at least one microchamber through the anterior opening and blocks the at least one posterior opening. Particles of interest can include cells, viruses, bacteria, amoeba, protozoa, paramecium, microparticles, nanoparticles, beads, artificial microswimmers, microorganisms, vesicles, and fragments thereof. The particles can be provided in any desired suspension fluid, including but not limited to water, cell growth media, serum, plasma, oil, and the like. In step 206, an isolating fluid is flowed into the at least one posterior port, such that the isolating fluid occupies each channel and isolates each microchamber. The isolation fluid can be any desired fluid. In certain embodiments, the isolation fluid is immiscible with a suspension fluid. Contemplated isolation fluids include but are not limited to water, cell growth media, serum, plasma, oils, gels, liquid metals, liquid polymers, glues, and the like.

The method steps illustrate the functionality of the microfluidic devices of the present invention. In a device that is free of particles, a suspension fluid entering an anterior port is free to flow into each of the fluidly connected channels and microchambers and out of a posterior port by way of unobstructed anterior openings, posterior openings, channel branches, and channel openings. A particle of interest that flows along the path of the suspension fluid and enters a microchamber through an anterior opening is guided to a posterior opening, whereupon a posterior opening selected for the particle of interest is too narrow to permit the particle to pass through. The particle thereby plugs or otherwise obstructs the posterior opening, thereby reducing or eliminating further flow into the microchamber in which the particle is occupying and preventing additional particles from entering the microchamber. Once an operator is satisfied that a requisite number of microchambers have been occupied by single particles, the flow of suspension fluid entering an anterior port can be halted. An isolating fluid can then be flowed through the posterior port towards the anterior port. Due to the size of the channel openings and the alignment of the channels with respect to the channel openings and anterior openings, the isolating fluid bypasses the microchambers to flush out any remaining suspension fluid in the channels. The isolating fluid, in occupying the volume of the channels, also blocks off the microchambers from each other, isolating each particle in its own fluid environment. Flowing fluids between anterior and posterior ports can be performed in any suitable manner. In some embodiments, fluid flow can be performed manually, such as with a syringe applying positive pressure through a first port or a negative pressure through a second port. In some embodiments, fluid flow can be performed using a pump, such as a positive pressure pump or a negative pressure pump.

The microfluidic devices are thereby capable of sequestering and studying particles of interest in complete isolation in any desired microenvironment. For example, cell or bacteria growth can be examined with minimal interference. Virus infection can be studied in individual cells or bacteria. Virus transmission can be studied between two or more cells or bacteria within a single microchamber.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out exemplary embodiments of the present invention, and are not to be construed as limiting in any way the remainder of the disclosure.

### Example 1: Towards Single-Cell Virology by High-Throughput Microfluidic Isolation and Pairing of Single-Cells

Viral pathogenesis is a process that alternates viral multiplication in host cells and transmission of viral particles to neighboring cells. To investigate both processes at the single-cell level, microfluidic devices were been developed for isolation of about 5000 single-cells or about 2000 pairs of two different cells with high tolerance to cell size and trapping parameters. By tracking and quantitating fluorescence from single-cells co-infected with GFP- and mCherry-fused viruses, new perspectives on viral replication masked by traditional population-based assays were unveiled: 1) host-dependence of the onset and virus-dependence of the yield and speed are observed in the multiplication of both coxsackievirus B3 (CVB3) and poliovirus (PV), but the host-dependence of the increase time only applied to CVB3 replication; 2) compared to CVB3, greater variances in the kinetic parameters of PV replication were observed, implying higher fidelity of RNA-dependent RNA polymerase (RdRp) encoded by CVB3. By pairing pre-infected cells and un-infected cells, unambiguous tracking of lytic and nonlytic spread of viral infection were realized, paving the way for a deeper understanding on virus-host interactions.

The following study demonstrates microfluidic platforms to replace conventionally-used multi-well plates for single-cell virology, leading throughput to a higher level and extending the application potential to a wider scope. In combination with live-cell imaging and quantitative analysis, proof-of principle investigations on dynamics of poliovirus (PV) replication have been successfully implemented. Recently, unique mechanism-determined signatures of different classes of antiviral compounds have also been revealed on a modified version of the platform (Liu W et al., bioRxiv, 2019, 606715). Nevertheless, these multi-layer PDMS devices suffer from complex fabrication and user-hostile manipulation procedures, hindering widespread adoption and use. Single-cell experiments with coxsackievirus B3 (CVB3) and PV infection were implemented and the results have been compared, providing new insights into the between-strain differences in viral replication. More importantly, this platform could be additionally adopted for cell pairing to study the spread of virus infection at the single-cell level. Covering both the viral multiplication in host cells and the transmission to un-infected cells, the microfluidic platform devices enable comprehensive analysis of the pathogenesis of viral diseases.

The materials and methods are now described.

HeLa S3 cells and HAP1 cells were both obtained from American Type Culture Collection (ATCC). HeLa S3 cells were maintained in DMEM/F12 (1:1) (Life Technologies) and HAP1 cells were maintained in IMDM (Life Technologies), both supplemented with 10% fetal bovine serum (FBS, Atlanta Biologicals) and 100 IU/mL penicillin—streptomycin (Corning), in a humidified atmosphere of 95% air and 5% CO2 at 37° C.

To obtain viruses used in this work, the wild-type (WT) and G64S poliovirus (PV) and coxsackievirus B3 (CVB3) cDNA were modified by insertion of GFP and mCherry sequences immediately 5′ of the open reading frame (ORF). A 3C protease site was also added to the 3′ end of the reporter sequences to ensure efficient cleavage of the fluorophore following translation of the viral RNA. PV and CVB3 cDNA were linearized with EcoRl and Clal respectively. Virus was produced from in vitro RNA and quantified for plaque forming units (PFU).

Each microfluidic device used in this work consisted of 5 sub-groups of 1200 (12*100) microchambers (FIG. 5). The molds for the single-cell devices and cell-pairing devices were respectively fabricated by multilayer lithography method. The first layer was 20 μm deep for the channels, while the second layer was 100 μm deep for the chambers. For both layers, SU8 photoresist was employed to deposit the patterns as designed in the photomasks (FIG. 5).

Microfluidic layers were fabricated from polydimethylsiloxane (PDMS,GE RTV615) with the molds. Premixed prepolymer and curing agent (ratio 10/1) were poured onto the mold and incubated at 75° C. for 45 min. Then the cured PDMS layer was peeled off, and inlets were punched as designed. The PDMS was sealed with a cleaned glass slide by oxygen plasma treatment.

HeLa S3 cells were mixed with virus at the MOI of 5 PFU/cell, and the mixture was shaken at 140 rpm for 30 min. Then the cells were centrifuged at 1000 rpm for 5 min, washed with PBS twice, and resuspended in normal culture medium. The cell suspensions were then infused to the inlets of a microfluidic device for single-cell trapping or cell pairing for 5 min. The channels were gently washed with culture medium to remove un-trapped cells. To isolate the single-cells/cell-pairs containing microchambers, FC70 oil (Sigma) with 2% (w/w) 008-FluoroSurfactant (RAN Biotechnologies) was infused into the inlets at the other end of the device.

For microscopic imaging, the microfluidic device was placed in the chamber of a stage top WSKM GM2000 incubation system (Tokai, Japan), which was adapted to a Nikon Eclipse Ti inverted microscope (Nikon, Japan). A ProScan II motorized flat top stage (Prior Scientific, USA) was quipped for automatic imaging. Bright-field and fluorescence images were acquired every 30 minutes from 1 hpi to 24 hpi using a CFI60 Plan Apochromat Lambda 10× objective and a Hamamatsu C11440 camera.

Fluorescence intensity and background intensity for each microchamber were extracted with a customized MATLAB script. (Fluorescence intensity-Background)/Background was calculated to represent the relative intensity. Wells containing two or more infected cells and showing auto-fluorescence or out-of-focus signals were excluded. As a result, the fluorescence intensity of each infected single-cell over time could be obtained. The maximum, slope, infection time, and start point were derived from previous studies (Guo F et al., Cell reports, 2017, 21(6):1692-1704; Caglar M U et al., PeerJ, 2018, 6:e4251).

The results are now described.

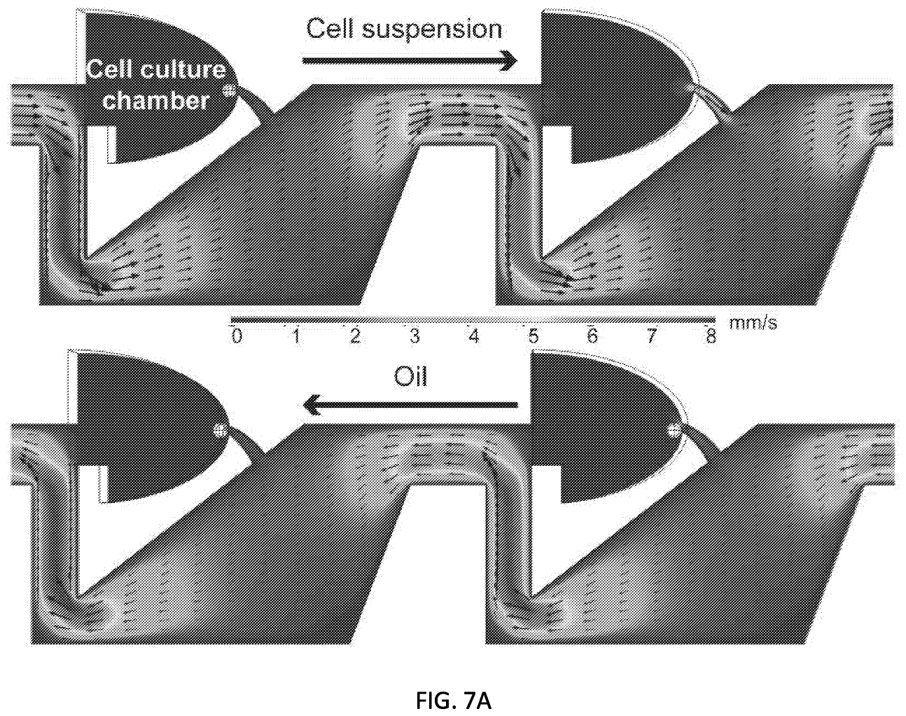

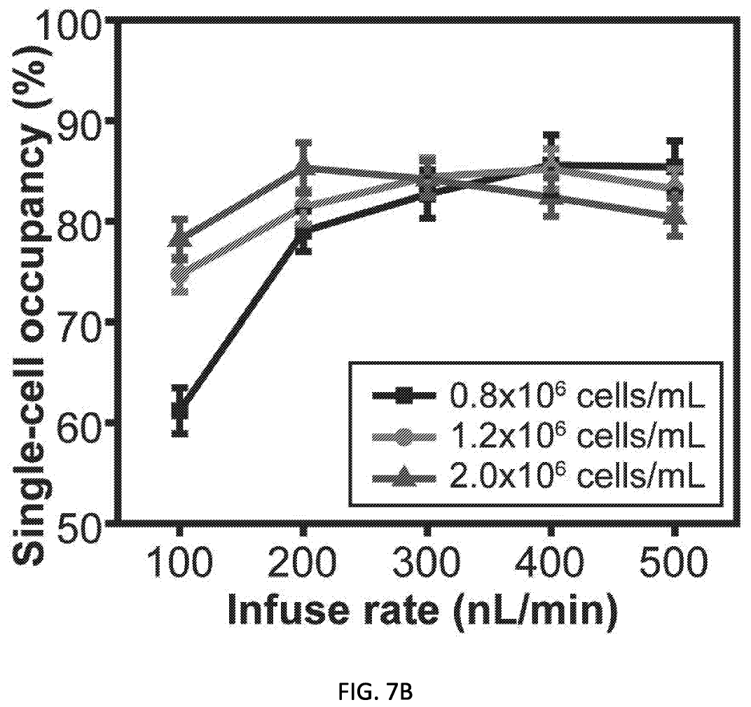

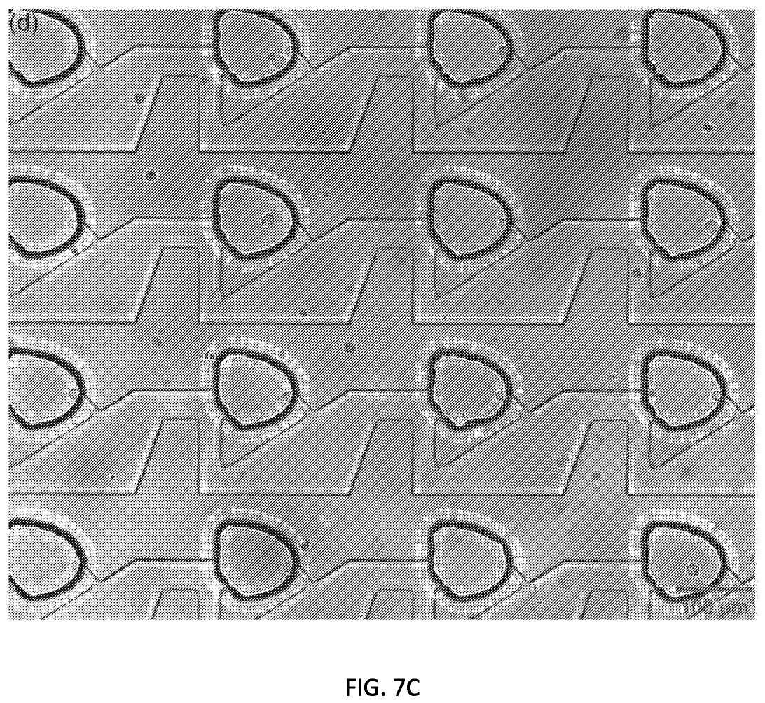

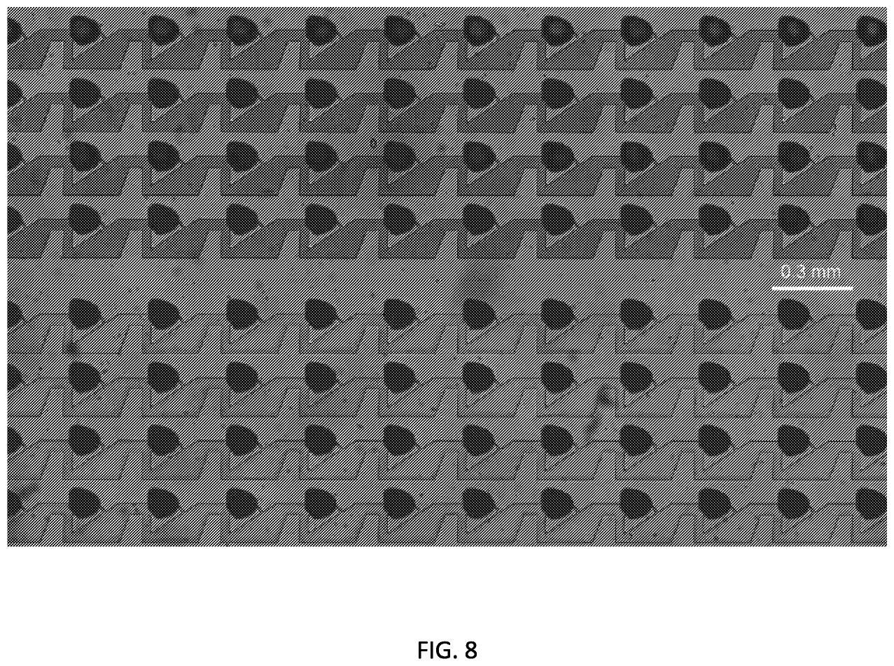

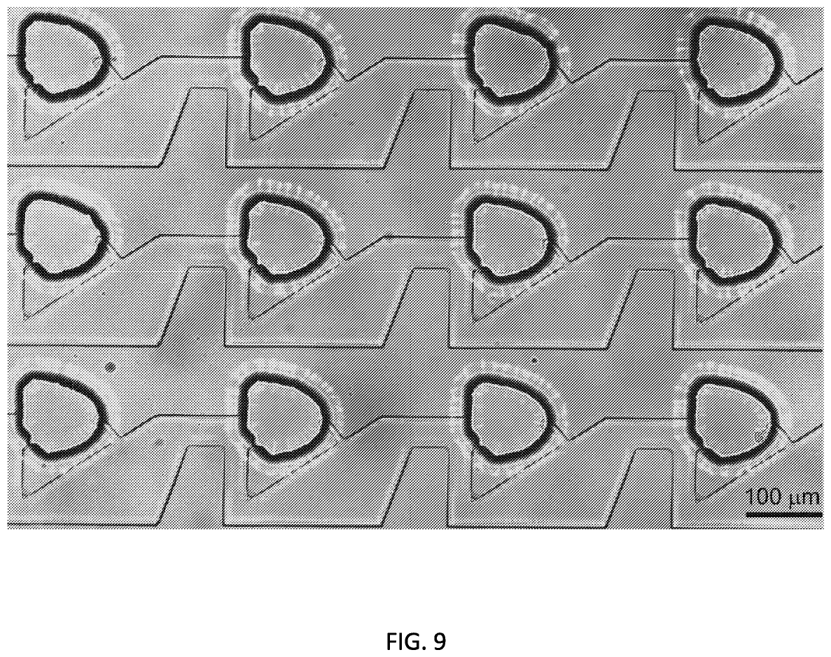

The geometry of the device containing 5 independent groups of 1200 microchambers for single-cell compartmentation and culturing is shown in FIG. 1 and FIG. 5. The devices can be repeatedly obtained with a mold, which could be either fabricated by soft lithography or 3D-printing. Simulation-guided design ensures that single-cells are driven into empty chambers but bypass occupied chambers (FIG. 7A). After trapping single-cells, the microchambers, each filled with nearly 1 nL culture medium, can be conveniently isolated by infusing FC-70 oil from the opposite direction (FIG. 7A and FIG. 8). Nearly 1000 single-cells can be isolated in each group with high tolerance to infuse rate, cell density, as well as cell size (FIG. 7B, FIG. 7C, and FIG. 9), endowing the device with universality and robustness to accommodate diversified biological studies.

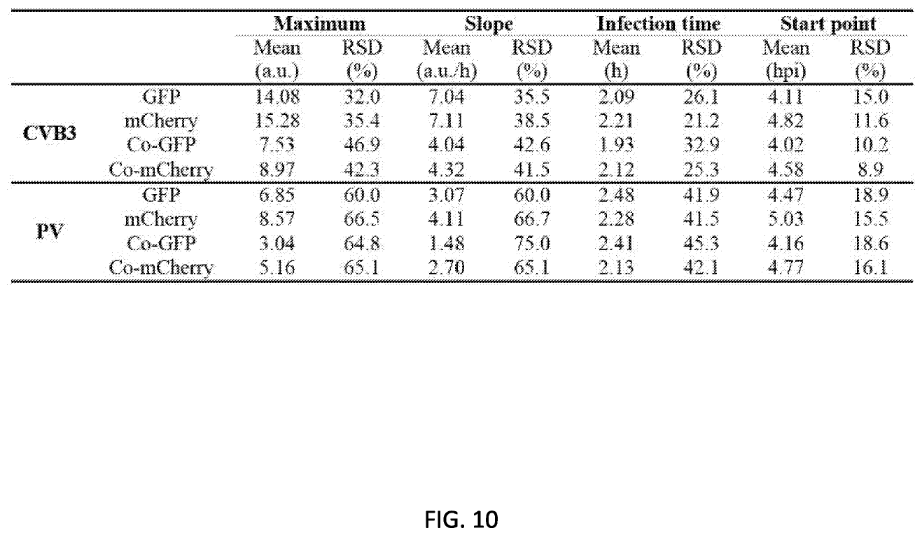

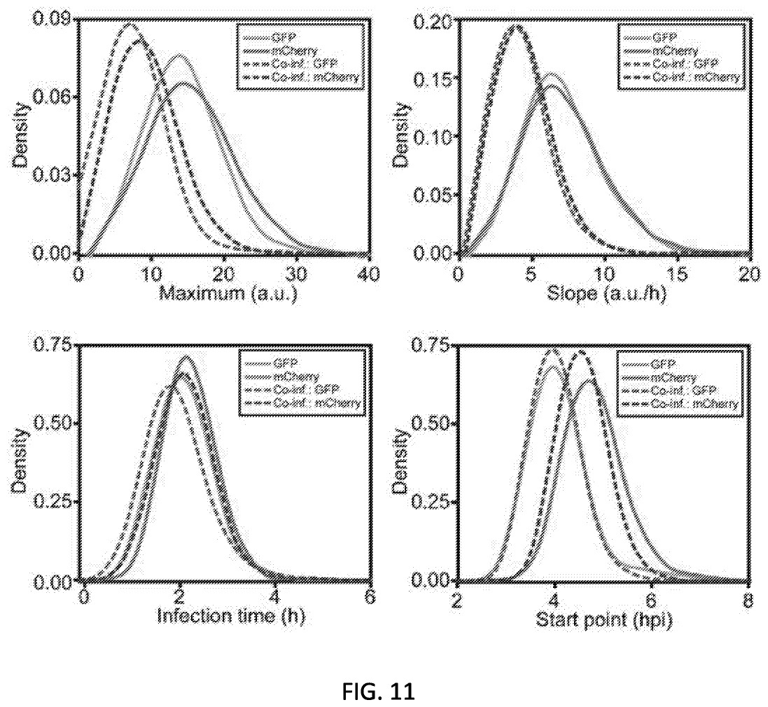

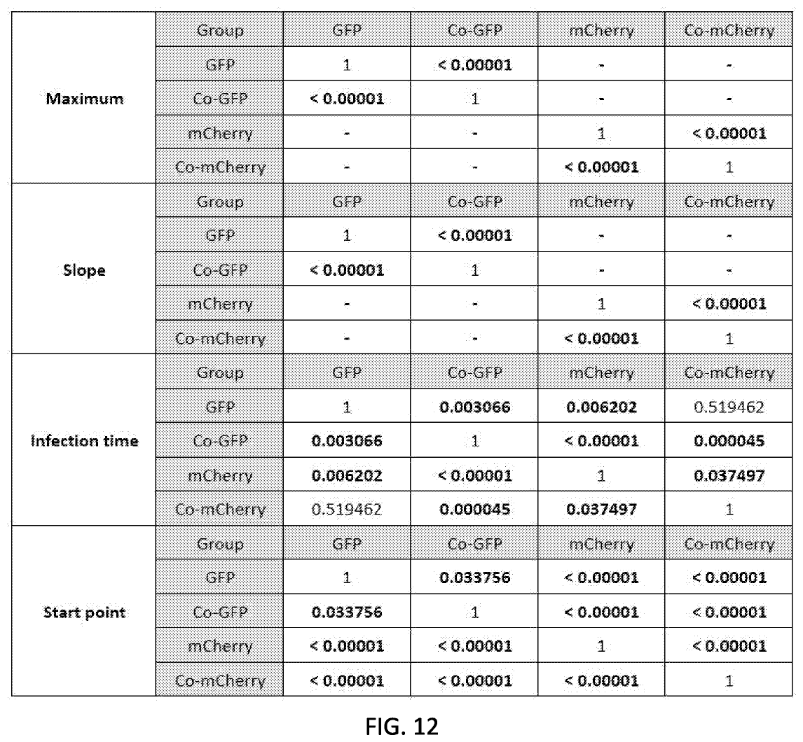

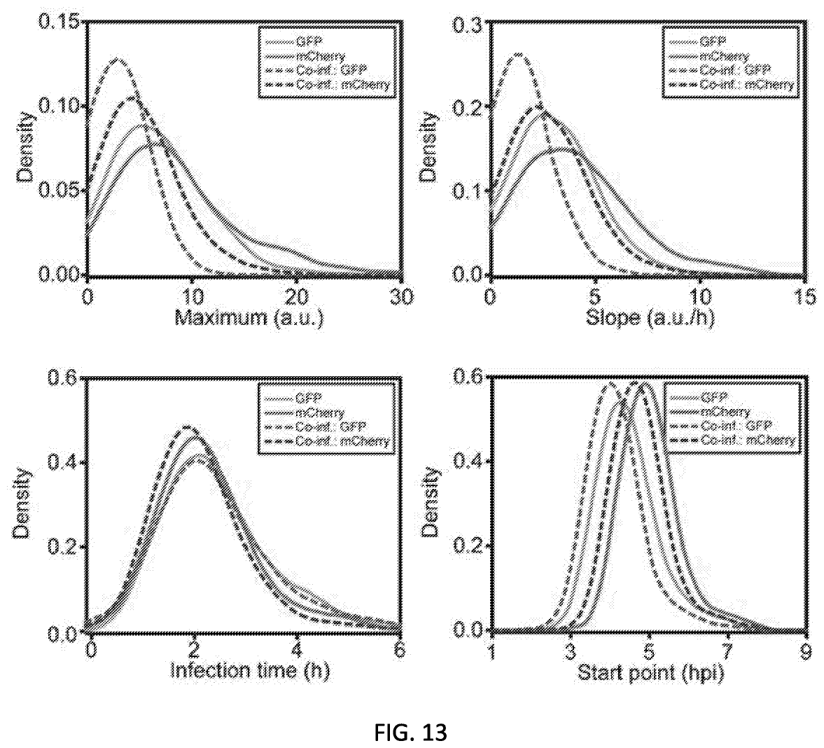

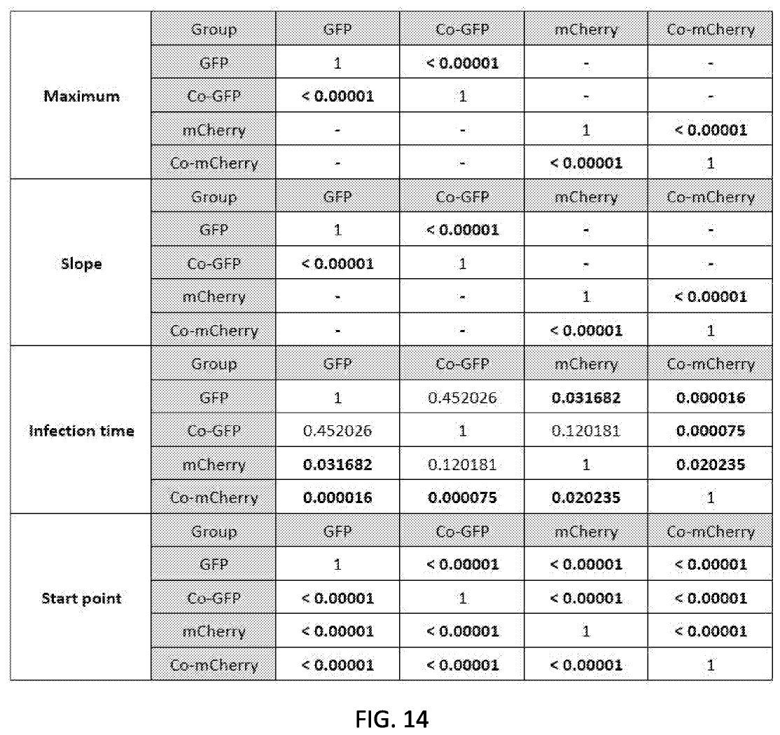

Co-infection experiments were then performed on the single-cell platform. HeLa S3 cells were loaded on-chip and imaged every 30 min between 1-24 hours-post-infection (hpi), after being infected with both CVB3-GFP and CVB3-mCherry, each at a multiplicity of infection (MOI) of 5 plaque-forming unit per cell (PFU/cell). For each co-infected single-cell, the GFP intensity and mCherry intensity over time were respectively extracted and processed by a previously reported model (Caglar M U et al., PeerJ, 2018, 6:e4251). Maximum, slope, infection time and start point were derived to describe the yield, speed, increase time and onset of the corresponding replication event in the host cell. Unsurprisingly, compared to cells solely infected by CVB3-GFP or CVB3-30 mCherry, co-infected cells host viral replications with slightly shortened infection time and earlier start point (FIG. 10, FIG. 11, and FIG. 12), due to the increase of combined MOI. Meanwhile, the maximum and slope for either GFP signal or mCherry signal are reduced by 40-50%, as the two types of viruses had to share the cellular resources in the co-infected cells. Similar phenomenon were also observed with PV (FIG. 10, FIG. 13, and FIG. 14).

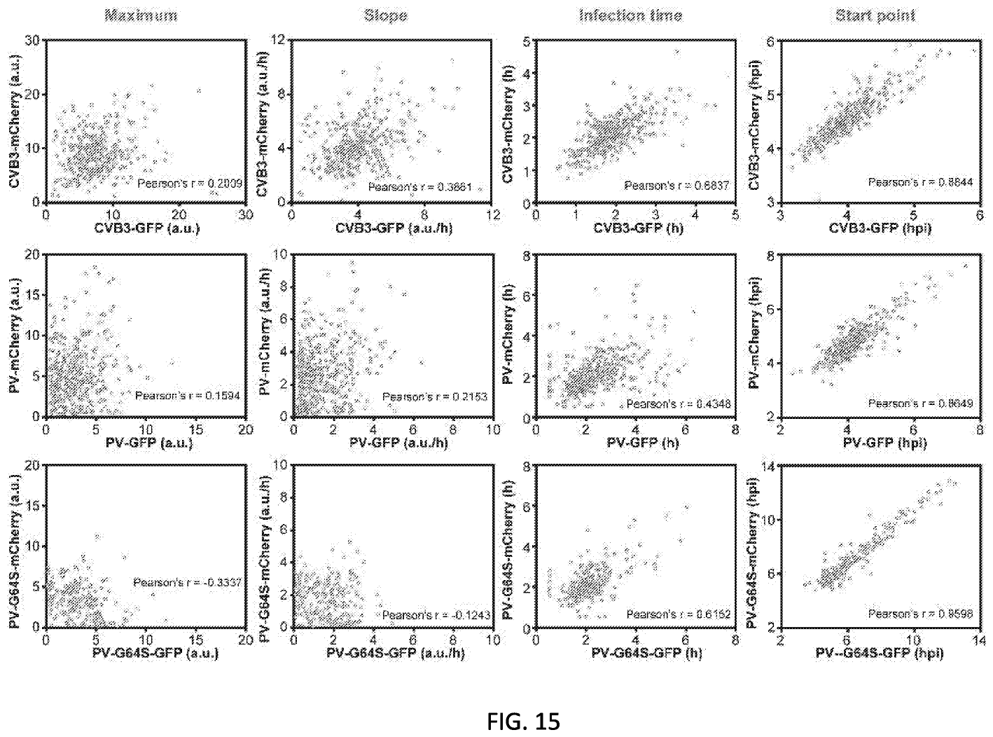

Correlations for these kinetics-related parameters were then investigated between the GFP signal and mCherry signal from the co-infected cells (FIG. 15). Parameters governed by the cell factors would exhibit strong correlations, while those primarily determined by the virus would not. As a result, for both CVB3 and PV, the yield and speed of the replication were virus-dependent but the onset was cell-dependent. However, a strong correlation in the infection time was found in CVB3 infection (Pearson's r=0.6837) but not in PV infection (Pearson's r=0.4348), which indicated that PV is able to evade some host responses related to the increase time but CVB3 is not. This difference might be owed to the higher fidelity of the RNA-dependent RNA polymerase (RdRP) encoded by CVB3 (Graci J D., Journal of virology, 2012, 86(5):2869-2873). For validation, a mutant with higher fidelity than the wild-type (WT) PV strain, PV-G64S, was tested with the same experiment (Pfeiffer J K., Proceedings of the National Academy of Sciences, 2003, 100(12):7289-7294). The correlation coefficient for infection time increases to 0.6152, demonstrating the importance of fidelity for this kinetic parameter.

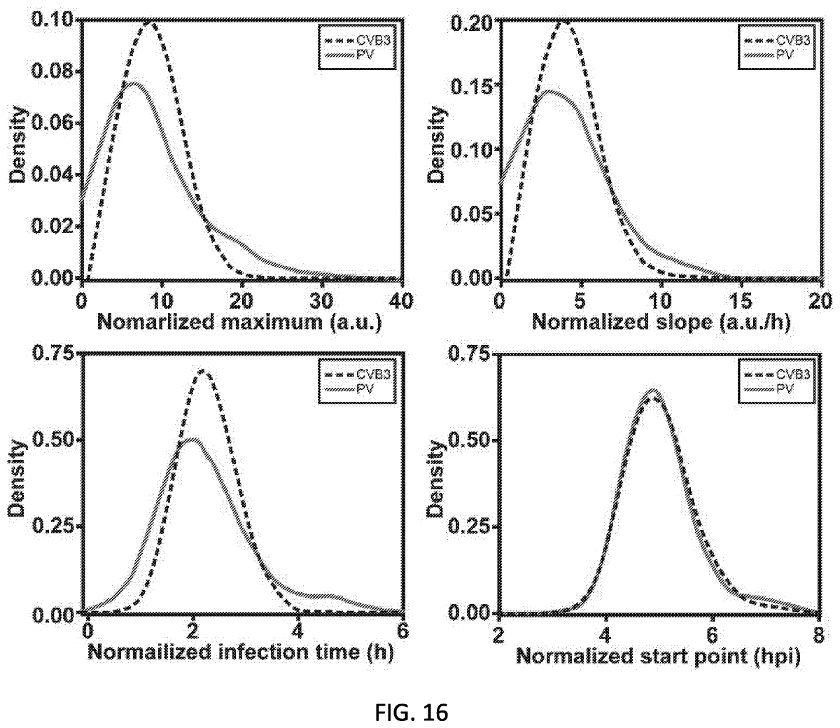

Another piece of evidence supporting the above inference arises from the comparison of the variances in the parameters between CVB3 and PV. As shown in FIG. 10 and FIG. 16, the kinetics of PV replication exhibited higher levels of heterogeneity in all the parameters, especially in maximum and slope, which were primarily determined by the virus.

Picornaviruses including CVB3 and PV have been well known as lytic viruses, however a few recent papers suggest that virus-containing vesicles could be released before the lysis of the infected cell, which makes the identification of donor and recipient cell pairs even more impracticable when being studied in bulk (Robinson S M., PLoS pathogens, 2014, 10(4):e1004045; Bird S W et al., Proceedings of the National Academy of Sciences, 2014, 111(36):13081-13086; Chen Y H et al., Cell, 2015, 160(4):619-630). A cell-pairing platform would thus offer unique opportunities for direct and unambiguous observation of the spread of viral infection between cells. Motivated by the high efficiency of the mechanism for single-cell trapping, an additional “trapping site” was added to each microchamber (FIG. 17A). Upon proper selection of cell density and infuse rate, cell pairs could be seeded in about two thirds of the microchambers (FIG. 17B).

Red dye-labelled HeLa S3 cells were infected with PV-GFP at a MOI of 5 PFU/cell, mixed with an equal number of uninfected cells, and pumped into a device, yielding about 2000 isolated pairs of an infected (donor) cell and an uninfected (recipient) cell. Being monitored by fluorescence microscopy, the dynamics of the viral replication in the donor cells and the spread to the recipient cells were recorded. Despite that around half of the recipient cells didn't turn green by 24 hpi, based on the time difference (Δt) between the emergence of green fluorescence in the recipient and the lysis of the donor cell, clear examples of nonlytic and lytic spread of PV infection were observed (FIG. 17C). A Δt of 1 hour was used as the cutoff since it takes at least 1 hour to see the GFP signal from a cell being infected (FIG. 17D). Although only being found in about 15% of the cell pairs, for the first time explicit and visual examples of the nonlytic spread of PV infection is shown. As the mechanism is still not fully understood to date, the microfluidic platform demonstrates its use as a powerful tool. The microfluidic platform can also be used with machine learning methods to separately extract fluorescence intensities of donor cells and recipient cells to enable further quantitative analysis.

In summary, microfluidic devices were developed for high throughput single-cell virology. Preliminary data shown in this work has revealed new insights based on the similarities and differences observed in CVB3 and PV replication dynamics in host cells. Meanwhile, unambiguous monitoring of the cell-to-cell transmission of PV infection was implemented on the cell-pairing device. Taking together, the versatile capability of the devices in studying cellular level pathogenesis of viral infection has been demonstrated, opening new avenues for mechanism elucidation and antiviral development. The present study represents a technical advance towards exploration of virus-host interaction at the single-cell level.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A microfluidic device for isolation of single particles, comprising:
a planar substrate having an anterior end, a posterior end, a top surface, a bottom surface, and a thickness in-between the top and bottom surface;
at least one anterior port and at least one posterior port extending from the top surface into the substrate;
at least one channel embedded within the substrate; and
at least one microchamber embedded within the substrate;
wherein each anterior port is fluidly connected to a posterior port by the at least one channel; and wherein the at least one microchamber is connected to a channel by an anterior opening and at least one posterior opening.

2. The device of claim 1, wherein the at least one microchamber comprises a shape that tapers posteriorly towards the at least one posterior opening.

3. The device of claim 1, wherein the at least one channel has a segment for each microchamber that is in direct alignment with the anterior opening of each microchamber.

4. The device of claim 1, wherein the at least one channel has a segment with a channel opening connected to each posterior opening of each microchamber by a channel branch.

5. The device of claim 4, wherein the segment with the channel opening is aligned along a first axis and the channel branch is aligned along a second axis, such that the first axis and the second axis are substantially orthogonal to each other.

6. The device of claim 1, wherein the at least one channel has a width between about 5 μm and 500 μm and a height or depth between about 5 μm and 50 μm.

7. The device of claim 1, wherein the at least one microchamber has a length, a width, and a height or depth that is each between about 20 μm and 500 μm.

8. The device of claim 1, wherein the anterior opening has a width between about 10 μm and 100 μm.

9. The device of claim 1, wherein the at least one posterior opening has a width between about 1 nm and 100 μm.

10. The device of claim 1, wherein the substrate is at least partially transparent or translucent.

11. The device of claim 1, wherein the at least one channel, the at least one microchamber, or both have one or more gradations within the thickness of the substrate.

12. The device of claim 1, wherein the at least one channel, the at least one microchamber, or both have an inner surface further comprising a surface treatment.

13. The device of claim 12, wherein the surface treatment includes an extracellular matrix material selected from the group consisting of: collagen, fibrin, fibrinogen, thrombin, elastin, laminin, fibronectin, vitronectin, hyaluronic acid, chondroitin 4-sulfate, chondroitin 6-sulfate, dermatan sulfate, heparin sulfate, vixapatin (VP12), heparin, and keratan sulfate, proteoglycans, chitin, chitosan, alginic acids, alginates, and combinations thereof.

14. The device of claim 12, wherein the surface treatment includes a drug selected from the group consisting of: analgesics, anesthetics, antifungals, antibiotics, anti-inflammatories, nonsteroidal anti-inflammatory drugs (NSAIDs), anthelmintics, antidotes, antiemetics, antihistamines, anti-cancer drugs, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, fluorescent nanoparticles such as nanodiamonds, sedatives, sulfonamides, stimulants, sympathomimetics, tranquilizers, urinary anti-infectives, vasoconstrictors, vasodilators, vitamins, xanthine derivatives, and combinations thereof.

15. The device of claim 12, wherein the surface treatment includes a capture agent selected from the group consisting of: antibodies, antigens, aptamers, affibodies, proteins, peptides, nucleic acids, carbon nanotubes, nanowires, magnetic beads, and fragments thereof.

16. A method of isolating single particles, comprising the steps of:
providing a microfluidic device comprising a substrate having at least one anterior port fluidly connected to at least one posterior port by one or more embedded channels, the substrate further comprising one or more embedded microchambers fluidly connected to each channel by an anterior opening and at least one posterior opening;
flowing a suspension fluid comprising at least one particle of interest in a population of particles into the at least one anterior port, such that a particle of interest enters at least one microchamber through the anterior opening and blocks the at least one posterior opening; and
flowing an isolating fluid into the at least one posterior port, such that the isolating fluid occupies each channel and isolates each microchamber.

17. The method of claim 16, wherein the suspension fluid, the isolating fluid, or both are flowed using a positive pressure or a negative pressure.

18. The method of claim 16, wherein the at least one particle of interest is selected from the group consisting of: cells, viruses, bacteria, amoeba, protozoa, paramecium, microparticles, nanoparticles, beads, microorganisms, vesicles, and fragments thereof.

19. The method of claim 16, wherein the suspension fluid is selected from the group consisting of: water, cell growth media, serum, plasma, and oil.

20. The method of claim 16, wherein the isolation fluid is selected from the group consisting of: oils, gels, liquid metals, liquid polymers, and glues.
